(19) United States
(12) Patent Application Publication
(10) Pub. No.: US 2005/0152953 A1
(43) Pub. Date: Jul. 14, 2005

(54) Aqueous medium for production of skin friendly non-woven materials

(76) Inventors: Christine Wild, Hilden (DE); Michael Neuss, Koeln (DE); Raymond Mathis, Duesseldorf (DE)

Correspondence Address: COGNIS CORPORATION;PATENT DEPARTMENT, 300 BROOKSIDE AVENUE, AMBLER, PA, 19002, US

(21) Appl. No.: 10/504,674

(22) PCT Filed: Feb. 6, 2003
(86) PCT No.: PCT/EP03/01155

(30) Foreign Application Priority Data
Feb. 15, 2002 (DE) 102 06 617.5

(51) Int. Cl. A61K 9/70

(52) U.S. Cl. 424/443

(57) ABSTRACT

A composition for use on hygiene articles containing: (a) from about 5 to 50% by weight of a first oil component having a melting point of from about 25 to 37° C.; (b) from about 5 to 50% by weight of a second oil component having a melting point of from about 40 to 60° C.; (c) optionally, a nonionic emulsifier; and (d) remainder, water, all weights being based on the total weight of the composition.

This invention relates to water-based compositions for finishing nonwovens, more particularly nonwovens which may be used in hygiene products.

In the production of hygiene articles, such as diapers or sanitary napkins, absorbent materials are used to take up aqueous liquids. In order to prevent direct contact with the absorbent material during wear and to increase wearing comfort, this material is enveloped in a thin water-permeable nonwoven. Such nonwovens are normally made from synthetic fibers, such as polyolefin or polyester fibers, because these fibers can be inexpensively produced, show good mechanical properties and are heat-resistant.

Nonwovens of the type in question used for hygiene articles are being increasingly finished with dermatologically compatible lotions in order generally to improve compatibility and wearing comfort. For example, DE 33 09 530 C1 describes a hygienic absorbent nonwoven which is impregnated with a skin-care preparation consisting of triglycerides and/or partial glycerides of coconut oil fatty acids containing 8 to 18 carbon atoms. To enable these preparations to be readily transferred from the nonwoven to the skin during wear, the triglyceride mixtures of DE 33 09 530 are selected so that they have a “rise” melting point of 35 to 40° C.

Another proposal for transferring skin-care preparations from hygiene articles to the skin during wear can be found in WO 96/16682. This document describes a diaper of which the inner nonwoven cover is finished with a lotion solid or semisolid at 20° C. which is transferred to the skin of the wearer during wear. These lotions contain 10 to 95% of a water-free emollient, which must be plastic or liquid at room temperature, and 5 to 90% of a so-called immobilizer with a melting point of at least 35° C., but preferably 40° C.

The main problem with the known lotions is their stability in storage. It is essential that the lotions have such a consistency at skin temperature, i.e. ca. 36 to 38° C., that they can be transferred without difficulty from the nonwoven to the skin. However, this temperature-controlled process can lead to problems when the hygiene products are stored at relatively high temperatures, for example above 30° C. The lotions are then often seen to “exude” from the nonwovens. Accordingly, the problem addressed by the present invention was to provide dermatologically compatible lotions for application to nonwovens for hygiene articles of which the stability in storage, particularly at relatively high temperatures, would have to be guaranteed.

In addition, the nonwoven would have to be permeable to liquids, for example in diapers, and hence would normally have been given a hydrophilic finish. Because of this, additional finishing with a generally hydrophobic dermatologically compatible lotion could reduce or distinctly impair the transport of liquids through the nonwoven into the absorbing materials.

In addition, the lotions would be required to be completely transferred from the nonwoven to the skin of the wearer and to perform other useful functions in the process, for example reducing odor emission or the growth of bacteria, fungi and yeasts. In principle, the lotions would of course have to be easy to apply to the nonwovens and to lend themselves to application by known finishing processes. The problems mentioned above could not be solved with the water-free lotions described in the prior art. More particularly, difficulties arise in the application of certain additives, for example zinc ricinoleate or chitosans, which only become active when applied from aqueous medium.

It has been found that suitable water-based compositions that satisfy the above-stated requirements are obtained by combining selected oil components.

The present invention relates firstly to compositions containing at least 5 to 50% by weight of a component a) melting at 25 to 37° C. selected from the group of paraffins, fatty acid esters, polyhydroxy fatty acid esters, fatty alcohols, alkoxylated fatty acid esters, alkoxylated fatty alcohols and mixtures of these compounds, 5 to 50% by weight of a component b) melting at 40 to 60° C. selected from the group of polyhydroxy fatty acid esters, C₁₄₋₂₂ fatty alcohols, C₁₂₋₂₂ fatty acids, alkoxylated derivatives of the fatty alcohols and esters and mixtures of these components and c) 5 to 25% by weight water.

The compositions according to the invention are emulsions or suspensions, preferably o/w or w/o emulsions.

In one preferred embodiment, the emulsions have a viscosity at 23° C. in the range from 100 to 10,000 mPa·s, preferably in the range from 500 to 5,000 mPa·s and more particularly in the range from 2,000 to 4,000 mPa·s (Brookfield RVF, spindle 5, 10 r.p.m., 23° C.). Besides special emulsifiers and selected oil components, the emulsions contain 25 to 45% by weight, preferably 25 to 40% by weight and more particularly 10 to 25% by weight water. The compositions according to the invention are preferably w/o emulsions.

Component a) may be selected from a number of compounds known to the expert, the melting range of these compounds having to be in the range from 25 to at most 37° C. Certain paraffins and also fatty acid esters and, more particularly, fatty alcohols may be used for this purpose. Suitable paraffins are preferably semisolid paraffins, such as soft paraffin, preferably petrolatum. Suitable fatty alcohols are, for example, dodecanol or ricinolyl alcohol to mention but one representative of the unsaturated fatty alcohols. The use of glycerides, preferably mixtures of partial glycerides and triglycerides which must have the required melting point of 25 to 37° C., is particularly suitable for the purposes of the present invention. Mixtures of glycerides of C₈₋₁₈ fatty acids are particularly preferred.

Glycerides are mono-, di and/or triesters of glycerol with fatty acids, i.e. for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid and erucic acid and technical mixtures thereof. They correspond to formula (I): 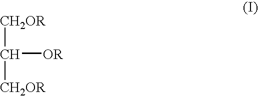
in which R is a group COR′, where R′ is a branched or unbranched, saturated or unsaturated C₆₋₂₂ alkyl group, and/or independently of one another represent hydrogen. Typical examples are lauric acid monoglyceride, lauric acid diglyceride, coconut oil fatty acid monoglyceride, coconut oil fatty acid triglyceride, palmitic acid monoglyceride, palmitic acid triglyceride, stearic acid monoglyceride, stearic acid diglyceride, isostearic acid monoglyceride, isostearic acid diglyceride, oleic acid monoglyceride, oleic acid diglyceride, tallow fatty acid monoglyceride, tallow fatty acid diglyceride, behenic acid monoglyceride, behenic acid diglyceride, erucic acid monoglyceride, erucic acid diglyceride and technical mixtures thereof which may still contain small amounts of triglyceride from the production process.

The use of emulsifier component c) is essential to the present invention. Glycerol partial esters with C₁₂₋₂₁ fatty acids, preferably glycerol monolaurate, are particularly suitable, as are polyvinyl sterolethers and—in a particularly preferred embodiment—polyglycerol poly-12-hydroxystearate. Polyol poly-12-hydrooxystearates are known substances which are marketed, for example, under the names of Dehymuls PWPH, Eumulgin VL 75 and Dehymuls SP11 by Cognis Deutschland GmbH. Further particulars of this compound can be found in European patent EP 0 766 661. The polyol component of these compounds can be derived from substances which contain at least 2, preferably 3 to 12 and more particularly 3 to 8 hydroxy groups and 2 to 12 carbon atoms. Typical examples are glycerol, polyglycerol, alkylene glycols, for example ethylene glycol, diethylene glycol and propylene glycol, methylol compounds, preferably trimethylolethane, trimethylolpropane, trimethylolbutane, pentaerythritol or dipentaerythritol, alkyl oligoglycosides containing 1 to 22, preferably 1 to 8 and more particularly 1 to 4 carbon atoms in the alkyl group, sugar alcohols containing 5 to 12 carbon atoms, for example sorbitol or mannitol, sugars containing 5 to 12 carbon atoms, preferably glycose or sucrose, and aminosugars, for example glucamine. The reaction product of poly-12-hydroxystearic acid with polyglycerol has proved to be particularly advantageous. The polyglycerol has the following composition: glycerols 5 to 35% by weight, diglycerols 15 to 40% by weight, triglycerols 10 to 35% by weight, tetraglycerols 5 to 20% by weight, pentaglycerols 2 to 10% by weight and, for the rest, oligoglycerols.

Besides components a) and b) and water, the compositions according to the invention may contain other ingredients, more particularly other emulsifiers, preferably nonionic emulsifiers. Nonionic emulsifiers are distinguished by their dermatological compatibility and mildness and by their favorable ecotoxicological properties. The use of a combination of nonionic emulsifiers leads to particularly fine-droplet emulsions, so that the stability of the composition is increased. The composition according to the invention contains the co-emulsifiers in a quantity of 0 to 15% by weight, preferably 1 to 10% by weight and more particularly 3 to 10% by weight, based on the weight of the composition.

The compositions according to the invention may additionally contain other typical ingredients, for example silicone waxes or polysiloxanes, in quantities of 1 to 6% by weight, preferably 1.5 to 5.5% by weight and more particularly 2 to 5% by weight. Polysiloxanes are known polymeric compounds which contain the following structure as monomer units: 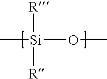
in which R″ and R′″ independently of one another represent hydrogen or an alkyl, cycloalkyl, aryl or alkenyl group. The siloxanes in question preferably have viscosities of 5 to 5,000 mPa·s at 37° C.

In addition, the compositions according to the invention may advantageously contain dermatologically compatible substances or skin-care substances, preferably in quantities of 0.1 to 10% by weight, more preferably in quantities of 1 to 8% by weight and most preferably in quantities of 2 to 6% by weight. Such ingredients may be, for example, bisabolol, alantoin and panthenol. Vitamins, preferably vitamin E, vitamin precursors and protein hydrolyzates may also be used. Also suitable are plant extracts, preferably of camomile, aloe vera, lime blossom, horse chestnut, green tea, oak bark, stinging nettle, hops, burdock root, horse willow, hawthorn, almond, pine needle, sandalwood, juniper, coconut, apricot, lemon, wheat, kiwi, melon, orange, grapefruit, sage, rosemary, birch, mallow, yarrow, thyme, balm, restharrow, coltsfoot, hibiscus, ginseng and ginger root. However, other skin-care substances may also be present, including in particular chitosan, zinc oxide and zinc ricinoleate.

In one particular embodiment of the invention, the emulsions may contain other auxiliaries and additives such as, for example, superfatting agents, thickeners, polymers, waxes, biogenic agents, deodorants, film formers, UV protection factors, antioxidants, hydrotropes, preservatives, insect repellents, self-tanning agents, solubilizers, stabilizers, perfume oils, dyes, germ inhibitors and the like.

Superfatting agents may be selected from such substances as, for example, lanolin and lecithin and also polyethoxylated or acylated lanolin and lecithin derivatives, polyol fatty acid esters, monoglycerides and fatty acid alkanolamides, the latter also acting as foam stabilizers.

Suitable thickeners are, for example, Aerosil types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol mono-esters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L/Grünau), quaternized wheat polypeptides, polyethyleneimine, cationic silicone polymers such as, for example, amodimethicone, copolymers of adipic acid and dimethylamino-hydroxypropyl diethylenetriamine (Cartaretine®/Sandoz), copolymers of acrylic acid with dimethyl diallyl ammonium chloride (Merquat® 550/Chemviron), polyaminopolyamides, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar® CBS, Jaguar®C-17, Jaguar® C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, α-hydroxycarboxylic acids, amino acids, ceramides, pseudoceramides, essential oils, plant extracts and vitamin complexes.

Suitable deodorizers are, for example, antiperspirants, such as aluminium chlorohydrates, aluminium/zirconium chlorohydrates and zinc salts. These antiperspirants are used for the production of perspiration-inhibiting and deodorizing preparations and probably act by partially blocking the sweat glands through the precipitation of proteins and/or polysaccharides. Besides the chlorohydrates, aluminium hydroxylactates and acidic aluminium/zirconium salts may also be used. For example, an aluminium chlorohydrate which corresponds to the formula [Al₂(OH)₅Cl].2.5H₂O and which is particularly preferred for the purposes of the invention is commercially available under the name of Locron® from Clariant GmbH. The aluminium/zirconium tetrachlorohydrex/glycine complexes marketed, for example, by Reheis under the name of Rezal® 36G are also preferably used in accordance with the invention. Other suitable deodorizers are esterase inhibitors, preferably trialkyl citrates, such as trimethyl citrate, tripropyl citrate, triisopropyl citrate, tributyl citrate and, in particular, triethyl citrate (Hydagen® C.A.T., Cognis Deutschland GmbH). Esterase inhibitors inhibit enzyme activity and thus reduce odor formation. The free acid is probably released through the cleavage of the citric acid ester, reducing the pH value of the skin to such an extent that the enzymes are inhibited. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester. Antibacterial agents which influence the germ flora and destroy or inhibit the growth of perspiration-decomposing bacteria, may also be present in the emulsions. Examples of such antibacterial agents are chitosan, phenoxyethanol and chlorhexidine gluconate. 5-Chloro-2-(2,4-dichlorophenoxy)-phenol, which is marketed under the name of Irgasan® by Ciba-Geigy of Basel, Switzerland, has also proved to be particularly effective.

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. The total percentage content of auxiliaries and additives may be from 1 to 50% by weight and is preferably from 5 to 40% by weight, based on the particular composition.

In addition, it can be of advantage to use stabilizers to stabilize the emulsion, for example glycerol or magnesium sulfate, preferably in quantities of 0.1 to at most 5% by weight and more particularly in quantities of 0.1 to 1.5% by weight.

The compositions may be produced by conventional cold or hot methods and are preferably produced by the phase inversion temperature method.

The present invention also relates to the use of the compositions described in the foregoing for the dermatologically compatible finishing of nonwovens.

The fleeces are materials known to the expert. The fleeces preferably used in accordance with the invention completely or partly contain polyolefins. Any known types of ethylene- or propylene-based polymers and copolymers are suitable for this purpose. Mixtures of pure polyolefins with copolymers are also suitable in principle. Polymers particularly suitable for the purposes of the teaching according to the invention are listed below: poly(ethylenes), such as HDPE (high-density polyethylene), LDPE (low-density polyethylene), VLDPE (very-low-density polyethylene), LLDPE (linear low-density polyethylene), MDPE (medium-density polyethylene), UHMPE (ultra high molecular polyethylene), VPE (crosslinked polyethylene), HPPE (high-pressure polyethylene); isotactic polypropylene; syndiotactic polypropylene; Metallocen-catalyzed polypropylene, high-impact polypropylene, random copolymers based on ethylene and propylene, block copolymers based on ethylene and propylene; EPM (poly[ethylene-co-propylene]); EPDM (poly[ethylene-co-propylene-co-unconjugated diene]). Other suitable polymers are: poly(styrene); poly(methylstyrene); poly(oxymethylene); Metallocen-catalyzed α-olefin or cycloolefin copolymers, such as norbornene/ethylene copolymers; copolymers containing at least 80% ethylene and/or styrene and less than 20% monomers, such as vinyl acetate, acrylates, methacrylates, acrylic acid, acrylonitrile, vinyl chloride. Examples of such polymers are: poly(ethylene-co-ethyl acrylate), poly(ethylene-co-vinyl acetate), poly(ethylene-co-vinyl chloride), poly(styrene-co-acrylonitrile). Also suitable are graft copolymers and polymer blends, i.e. mixtures of polymers in which the above-mentioned polymers inter alia are present, for example polymer blends based on polyethylene and polypropylene.

Homopolymers and copolymers based on ethylene and propylene are particularly preferred for the purposes of the present invention. In one embodiment of the present invention, therefore, polyethylene on its own is used as the polyolefin; in another embodiment, polypropylene on its own is used as the polyolefin and, in a further embodiment, ethylene/propylene copolymers are used as the polyolefin.

In a most particularly preferred embodiment of the invention, component a) is polypropylene.

The present invention also relates to the use of the hydrophilicized polyolefin- or polyester-based fibers wettable by aqueous media obtained by the process described above for the production of sheet-form textiles, preferably nonwovens. In a particularly preferred embodiment, these sheet-form textiles are intended for use in diapers.

### EXAMPLES

The following compositions according to the invention were prepared:

Emulsion 1

51% by weight partial glyceride/triglyceride mixture based on C₁₄/C₁₆ fatty acids, melting range: 33.0-35.5° C.
17% by weight partial glyceride/triglyceride mixture based on C₁₄/C₆ fatty acids, melting range: 40.0-42.0° C.
10% by weight polyhydroxystearic acid polyglycerol ester
1.45% by weight glycerol (86%)
0.30% by weight MgSO₄×7H₂O
rest water
   Emulsion 2
48% by weight partial glyceride/triglyceride mixture based on C₁₄/C₁₆ fatty acids, melting range: 33.0-35.5° C.
15% by weight partial glyceride/triglyceride mixture based on C₁₄/C₆ fatty acids, melting range: 40.0-42.0° C.
10% by weight polyhydroxystearic acid polyglycerol ester
5% by weight ZnO
1.45% by weight glycerol (86%)
rest water
   Emulsion 3
40% by weight partial glyceride/triglyceride mixture based on C₁₄/C₁₆ fatty acids, melting range: 33.0-35.5° C.
15% by weight partial glyceride/triglyceride mixture based on C₁₄/C₆ fatty acids, melting range: 40.0-42.0° C.
13% by weight polyvinyl stearyl ether
1.45% by weight glycerol (86%)
0.30% by weight MgSO₄×7H₂O
rest water

Using a Nordson slotcoater, the emulsions were applied to PP nonwovens in concentrations of 10 and 30 mg/l.

1-11. (canceled)

12. A composition comprising:
(a) from about 5 to 50% by weight of a first oil component having a melting point of from about 25 to 37° C.;
(b) from about 5 to 50% by weight of a second oil component having a melting point of from about 40 to 60° C.;
(c) optionally, a nonionic emulsifier; and
(d) remainder, water, all weights being based on the total weight of the composition.

13. The composition of claim 12 wherein (a) is present in an amount of from about 15 to 60% by weight, based on the weight of the composition.

14. The composition of claim 12 wherein (b) is present in an amount of from about 10 to 60% by weight, based on the weight of the composition.

15. The composition of claim 12 wherein the composition has a viscosity, at 23° C., of from about 100 to 10,000 mPa·s.

16. The composition of claim 12 wherein the composition has a viscosity, at 23° C., of from about 500 to 5,000 mPa·s.

17. The composition of claim 12 wherein the composition has a viscosity, at 23° C., of from about 2000 to 4,000 mPa·s.

18. The composition of claim 12 wherein (c) is present in the composition in an amount of from about 3 to 10% by weight, based on the weight of the composition.

19. A non-woven article having a composition containing:
(a) from about 5 to 50% by weight of a first oil component having a melting point of from about 25 to 37° C.;
(b) from about 5 to 50% by weight of a second oil component having a melting point of from about 40 to 60° C.;
(c) optionally, a nonionic emulsifier; and
(d) remainder, water, all weights being based on the total weight of the composition.

20. The article of claim 19 wherein (a) is present in an amount of from about 15 to 60% by weight, based on the weight of the composition.

21. The article of claim 19 wherein (b) is present in an amount of from about 10 to 60% by weight, based on the weight of the composition.

22. The article of claim 19 wherein the composition has a viscosity, at 23° C., of from about 100 to 10,000 mPa·s.

23. The article of claim 19 wherein the composition has a viscosity, at 23° C., of from about 500 to 5,000 mPa·s.

24. The article of claim 19 wherein the composition has a viscosity, at 23° C., of from about 2000 to 4,000 mPa·s.

25. The article of claim 19 wherein (c) is present in the composition in an amount of from about 3 to 10% by weight, based on the weight of the composition.

26. The article of claim 19 wherein the article is a hygiene article selected from the group consisting of a diaper and a sanitary napkin.
